(19) United States
(12) Patent Application Publication
(10) Pub. No.: US 2009/0248059 A1
(43) Pub. Date: Oct. 1, 2009

(54) Embolectomy Catheter

(76) Inventor: Hesham Morsi, Houston, TX (US)

Correspondence Address: LAW OFFICE OF DAVID MCEWING, P.O. BOX 231324, HOUSTON, TX, 77023, US

(21) Appl. No.: 12/180,542

(22) Filed: Jul. 27, 2008

Related U.S. Application Data
(60) Provisional application No. 61/070,726, filed on Mar. 25, 2008.

(51) Int. Cl. A61M 25/10

(52) U.S. Cl. 606/200

(57) ABSTRACT

The device subject of this disclosure is a catheter conveying inflatable components that control the opening and closing of a net component. The catheter is maneuvered through the tortuous and stenotic cerebral circulatory system to an embolism. The positioning of the catheter may be guided by radio opaque markers. The inflatable component carrying the net is placed into the embolism. The balloons are inflated thereby expanding the net into the embolism. The limit of expansion is controlled by the dimensions of the net. As the net expands, fenestrations or the spacing of a wire mesh comprising the net enlarge, thereby allowing the embolism to become entrapped within the net. The balloons are deflated thereby enclosing the embolism within the net. The device can be retract into a guide catheter and withdrawn from the circulatory system.

### RELATED APPLICATIONS

This application claims the benefit of and priority to provisional application Ser. No. US 61/070,726; entitled ANGIONET EMBOLECTOMY CATHETER; filed Mar. 25, 2008. This application is incorporated by reference herein.

### BACKGROUND OF INVENTION

1. Field of the Invention

The present invention relates generally to catheters and more particularly, to catheters for use as embolectomy catheters for treating diseases including but not limited to stroke.

2. Related Technology

Fixed dimensioned mechanical devices are known for attempting to retrieve or entrap clots. Disadvantages include insertion and withdrawal of these fixed dimensioned devices through the tortuous or stenotic vessels of the cerebral blood system. The devices may be made of stainless steel or similar firm material. They may be dimensioned to have a fixed diameter approximately as large as the interior vessel wall. These devices also have limitations in retaining all of the clot material in maneuvering the devices through the vessel system. It is possible that use of these devices will result in perforation of the vessel wall. It is also possible that portions of the embolus may break free from the device and cause further injury to the patient. These devices require greater time to utilize than the device subject of this disclosure. For example, it may not be possible to timely retrieve a portion of the embolism that has broken free of the existing retrieval device

### SUMMARY OF THE INVENTION

The present invention relates to the use of a segmented balloon catheter (at least 2 balloon segments) to deploy a net through and around the clot, forcing the clot inside the net, then deflating the balloons either partially or completely to collapse the net on the clot, then removing the net and the trapped clot into the guiding catheter. The balloons and net are made of soft material that will not damage the vessel walls. The embolectomy catheter is maneuvered through the cerebral vessel system in a collapsed state, thereby having a small diameter and flexibility that allows navigation through the cerebral circulatory system with less disturbance of the blood flow and rate.

### SUMMARY OF DRAWINGS

The accompanying drawings, which are incorporated in and constitute a part of the specification, illustrate preferred embodiments of the invention. These drawings, together with the general description of the invention given above and the detailed description of the preferred embodiments given below, serve to explain the principles of the invention.

FIG. 1 illustrates the segmented balloon catheter and the collapsed net in its deflated deliverable form.

FIG. 2 illustrates a cross sectional view of the catheter and the net in its deflated deliverable form.

FIG. 3 illustrates the segmented balloon catheter and the net in its fully inflated and deployed form with the longitudinal fenestrations.

FIG. 4 illustrates the segmented balloon catheter and the net in its fully inflated and deployed form with the horizontal fenestrations.

FIG. 5 illustrates cross sectional view of the catheter and the net in its deflated deliverable form.

FIG. 6 illustrates the segmented balloon catheter and the collapsed net in its deflated deliverable form advanced over the wire through the clot.

FIG. 7 illustrates the segmented balloon catheter partially inflated and the net partially deployed through the clot.

FIG. 8 illustrates the segmented balloon catheter fully inflated and the net fully deployed through and around the clot.

FIG. 9 illustrates the segmented balloon catheter deflated and the net collapsed over the clot.

FIG. 10 illustrates the withdrawing step of the segmented balloon catheter deflated and the net collapsed over the clot and retracted into the guiding catheter.

FIG. 11 illustrates the segmented balloon catheter utilizing a mesh of micro-threads as the entrapping mechanism.

### DETAILED DESCRIPTION OF INVENTION

The following discussion is directed to various embodiments of the invention. Although one or more of these embodiments may be preferred, the embodiments disclosed should not be interpreted, or otherwise used, as limiting the scope of the disclosure. In addition, one skilled in the art will understand that the following description has broad application, and the discussion of any embodiment is meant only to be exemplary of that embodiment, and not intended to suggest that the scope of the disclosure is limited to that embodiment.

Certain terms that are used throughout the following description refer to particular features or components. As one skilled in the art will appreciate, different persons may refer to the same feature or component by different names. This document does not intend to distinguish between components or features that differ in name but not function. The drawing figures are not necessarily to scale. Certain features and components herein may be shown exaggerated in scale or in somewhat schematic form and some details of conventional elements may not be shown in interest of clarity and conciseness.

In the following discussion the terms “including” and “comprising” are used in an open-ended fashion, and thus should be interpreted to mean “including, but not limited to . . . . ” Also, the term “distal” is intended to refer to positions relatively away from the operator of the catheter when it is in use, while the term “proximal” is intended to refer to positions relatively near the operator when the catheter is in use’. As a result, the distal end of a device is relatively near the embolus as compared to the proximal end of the device, which is relatively away from the embolus. An embolus is sometimes referred to herein as a “clot”. In addition, the term “radial” is intended to refer to movement toward or away from the longitudinal central axis of the catheter. The term “axial” is meant to refer to positions lengthwise along the central axis of the catheter.”

The device of the invention may be maneuvered through the tortuous cerebral circulatory system. The components are sized for the narrow, twisting path comprising thin wall vessels. The materials of the device may be compliant to minimize injury to the vessel walls. In one embodiment the length of the net is between 10 to 20 millimeters.

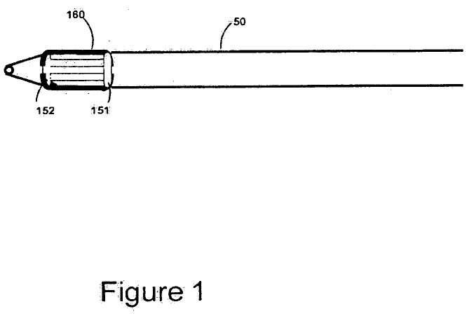

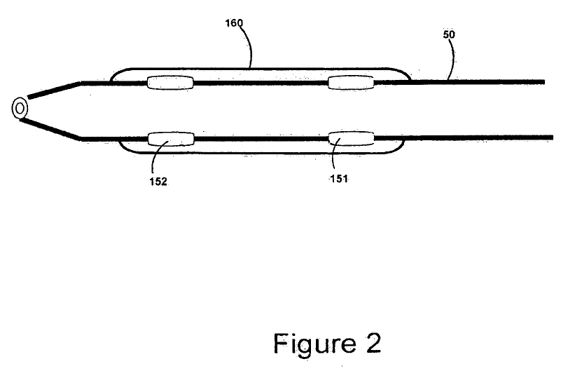

In an embodiment, the present embolectomy catheter comprises a first catheter having a proximal and a distal end, a fluid inlet at the proximal end, and discrete expandable segments (at least two balloons) placed along the distal shaft of the catheter. Inflation of the balloons with fluid deploys a tubular shaped net component that radially expands in a controlled manner to entrap embolus as discussed below. FIG. 1 illustrates the delivery catheter 50 transporting two balloons 151, 152 (uninflated). Also illustrated is one embodiment of a net 160 comprised of micro thread oriented longitudinally parallel to the catheter. FIG. 2 is a cross-sectional detail of the catheter 50 as conveyed through a artery (not shown). The balloons 151, 152 are deflated and the net 160 is proximate to the outer catheter wall. It will be appreciated that the balloons circumvent the catheter. The relationship of the net to the catheter and balloons is also demonstrated. Also illustrated is the hole at the distal tip of the catheter through which the guide wire passes. It will be noted that there is an area of the catheter that is bounded by the two balloons. It will be further noted that portions of the net may extend past this area in the proximate and distal direction. It is this portion of the net that the net cone segment may be formed as discussed further herein.

The net may have proximal and distal cone shaped ends. In addition, the inflatable segments are expanded through dispersal of fluid that flows through a tube in the catheter from the first catheter's proximal inlet. The inflatable segments (balloon) can be contracted through withdrawal of fluid from the first catheter's proximal inlet. (The first catheter is sometimes referred to herein as the delivery catheter.) The device may include a second and larger catheter, i.e., guiding catheter. When fully deflated, the first catheter containing the fluid tube, guide wire, balloons and net, can fit through the guiding catheter. In some cases, only the cone shaped portion of the collapsed net, filled with embolic material, will fit into the opening of the guiding catheter.

In another embodiment, the first catheter may contain one or more suction ports within the area between the first and second inflation segments. These ports draw fluid and embolismic matter into and through the net where it can be removed by deflation of the inflation segments and retraction of the catheter.

An additional embodiment includes the net which is cylindrical or tubular shaped and sized to the blocked artery. The outer diameter of the net may be dimensioned to match the inner diameter of the cerebral vessel containing the embolus. This includes selection of a balloon net combination that has both the appropriate inflatable diameter for the vessel and length to ensnare the embolus.

In one embodiment, the distal balloon may have a smaller diameter than the proximal balloon. This may adopt the shape of the net to the narrowing of the vessel, e.g., an artery.

It will be appreciated that when the net and balloon device is maneuvered to the site of the embolism, the balloons are deflated and the net is folded up or wrapped around the first catheter or balloon within the delivery catheter and have a minimum diameter. See FIGS. 1 and 2. This allows easier maneuvering of the device through the tortuous and stenotic cerebral circulatory system to the location of the embolism. Multiple fenestrations are distributed around the radial circumference of the net either along the longitudinal axis, horizontal axis, in a diagonal pattern (helical pattern) or any variation. Inflation of the balloons causes expansion or deployment of the net. The expansion of the net causes the fenestrations to further open facilitating capture of embolus. The shape of these fenestrations can be linear, round, oval, rectangular, diamond, serpentine or any other shape or combination of shapes.

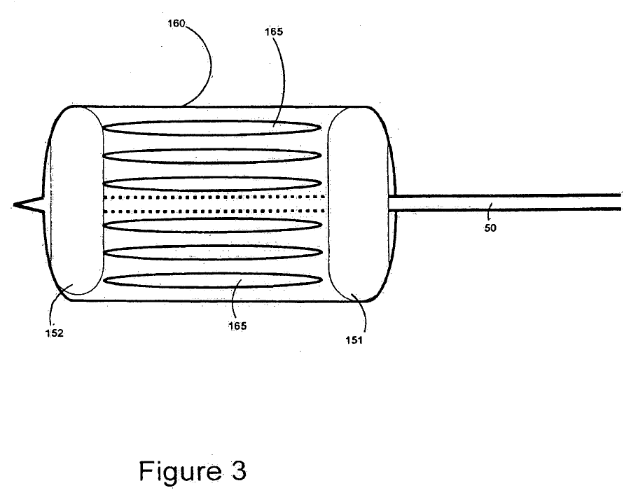

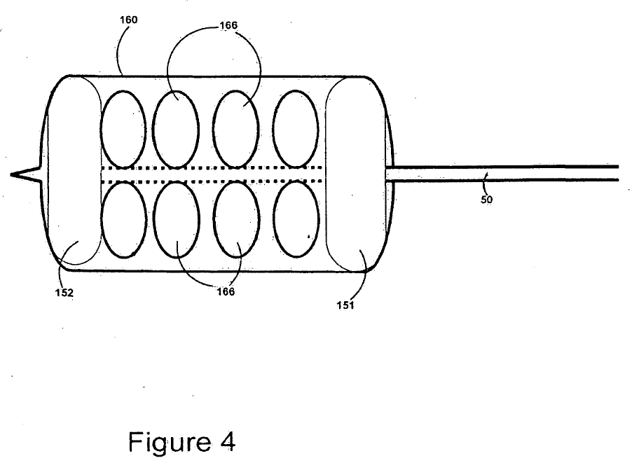

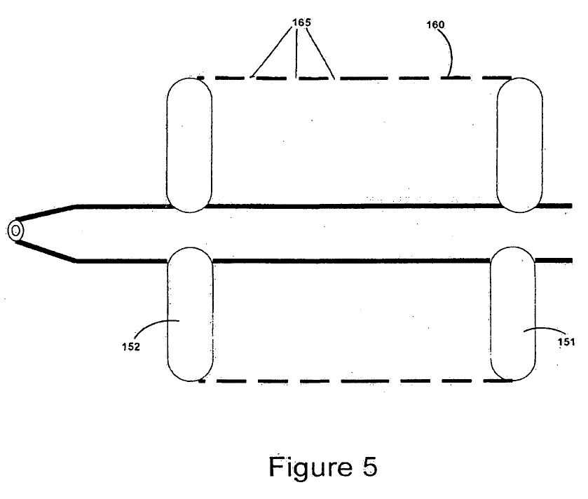

FIG. 3 illustrates a catheter 50 wherein the balloons 151, 152 are inflated. The net 160 is extended and longitudinal fenestrations 165 are illustrated to be open. FIG. 4 illustrates the catheter 50, inflated balloons 151, 152, deployed net with fenestrations 165 in a horizontal orientation. The fenestrations may also be in a helical pattern around the tube shaped net. This is sometimes termed diagonal. FIG. 5 illustrates a cross sectional view of the delivery catheter with the inflated balloons 151, 152. Also shown are the boundary of the net 160 and the fenestrations of the net 165. Note the net has been extended from the wall of the catheter.

The net material may be compliant, or semi-elastic in the middle of its length and inelastic, i.e., non-compliant, at the cone shaped ends of the net and its fitting over the balloons. The materials of this net can be non-compliant or semi-compliant biocompatible materials such as polytetrafluoroethylene (PTFE) or biocompatible small diameter micro threads. These material properties permit the inflated device to be dimensioned to the interior diameter of the vessel wall and without strain to the vessel wall caused by over inflation of the balloons. Stated differently, in one embodiment, the inelastic ends of the net covering the balloons prevent the balloons from over-inflating and detrimentally pressing against the vessel wall.

As the ends of the net are deployed over the fluid expanding balloons, the middle segment of the netting is also stretched and lifted away from the delivery catheter. See FIG. 5. Recall the net material between the balloons may be non compliant or semi-compliant.

In one embodiment, the netting is fabricated using small diameter micro-threads. This device can be constructed so that pulling upon a single thread (or group of threads) extending the length of the catheter can constrict the netting by pulling upon this extended thread. In one embodiment, this can be achieved by threads being interlaced through holes or under small protrusions within the delivery catheter. Pulling on one or more threads with tighten the mesh of micro-fibers. The micro-thread forms a mesh between the balloons. The openings within this mesh enlarge with inflation of the balloons and again shrink when the balloons are deflated.

The micro-threads may be woven or unwoven. In a preferred embodiment, the micro-threads are inelastic and non compliant. Alternatively the threads are semi-compliant within the middle section between the inflatable balloons. It will be appreciated that this construction will restrict the expansion of the balloons but facilitate the entry and entrapment of the embolism.

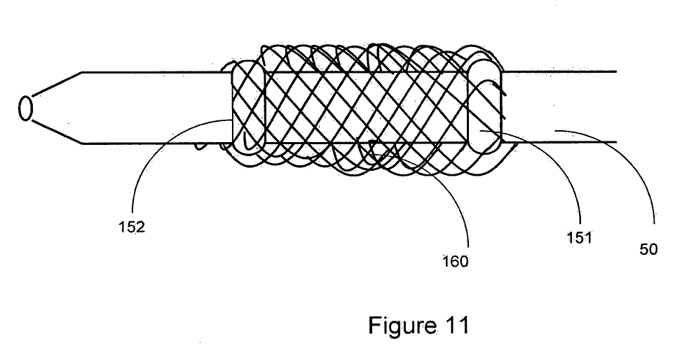

In another embodiment, the micro-threads may be helically wound, woven in a tubular knit or braided about the delivery catheter. FIG. 11 illustrates net constructed from micro-threads 161 wound in a helical pattern over the balloons 151, 152. Also illustrated is the delivery catheter 50. When the balloons are inflated, the openings of the mesh of threads will enlarge. These enlarged openings will facilitate entrapment of the embolus within the net. In yet another embodiment, the micro-threads may be installed over each balloon in a manner of orientation parallel to the delivery catheter. As the circumference of the inflating balloons increases, the spacing between the radially positioned threads also increases. See FIG. 1.

The net may be tapered at both ends to a cone shape. These tapered ends of the net may be fused to the first catheter shaft proximate to the proximal inflatable segment and distal to the distal inflatable segment. The cone shape provides additional carrying space for embolic material and may facilitate the retraction of the net into the second guiding catheter due to the tapered shape. In another embodiment, the net may be attached to the outer circumference of the balloon.

In another embodiment, the diameter of the net can be reduced by twisting the net around the first catheter.

It will be appreciated that the embolus, formed in an aqueous environment, may have a generally soft consistency which facilitates passage of the first catheter, net and at least one deflated balloon through the embolus. Further, the relative softness of the embolus allows portions to be pressed through the fenestrations of the net. The embolus can also be compressed as the net assumes a smaller diameter with deflation of the balloons. The cone shaped end of the net, now containing embolic material, can now be pulled into the second, larger diameter guiding catheter. Upon contraction or partial contraction of the expandable segments and the net, some or the entire first catheter may be drawn into the guiding catheter.

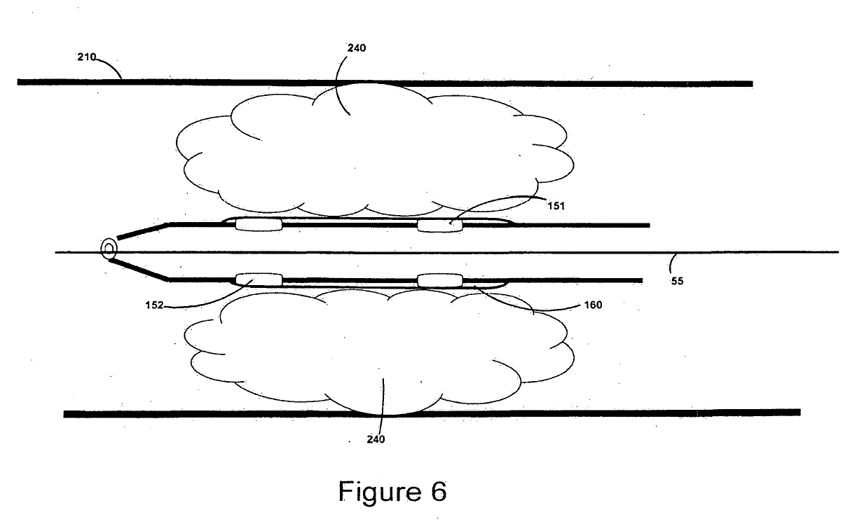

FIG. 6 illustrates a cross sectional view of the passage of the delivery catheter through the embolus 240 using the guide wire 55 extending through the distal end of the catheter. The guide wire is used for maneuvering the catheter through the circulatory system. The balloons 151, 152 are deflated and the net is proximate to the outer diameter of the catheter. Also illustrated is the vessel wall 210.

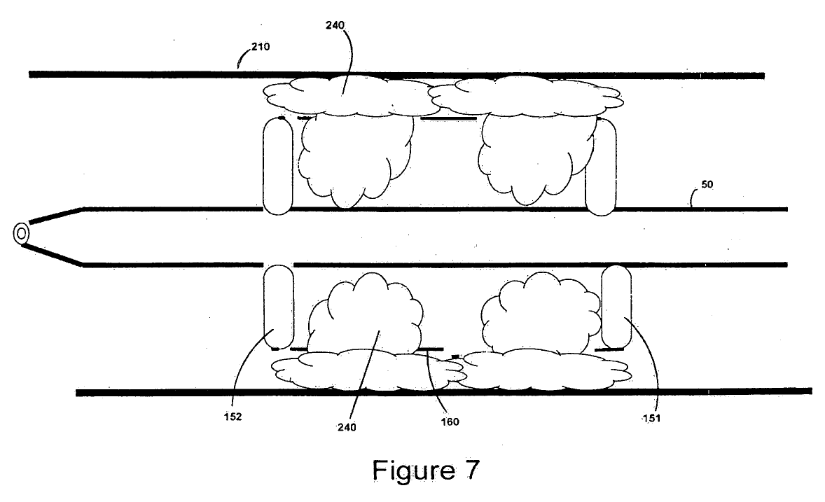

FIG. 7 illustrates the beginning of the balloon 151,152 inflation with the resulting radial stretching or expansion of the net 160. Some of the embolus is shown being pressed through the fenestrations of the net. This entrapped embolus remains between the outer diameter of the net and the catheter 50.

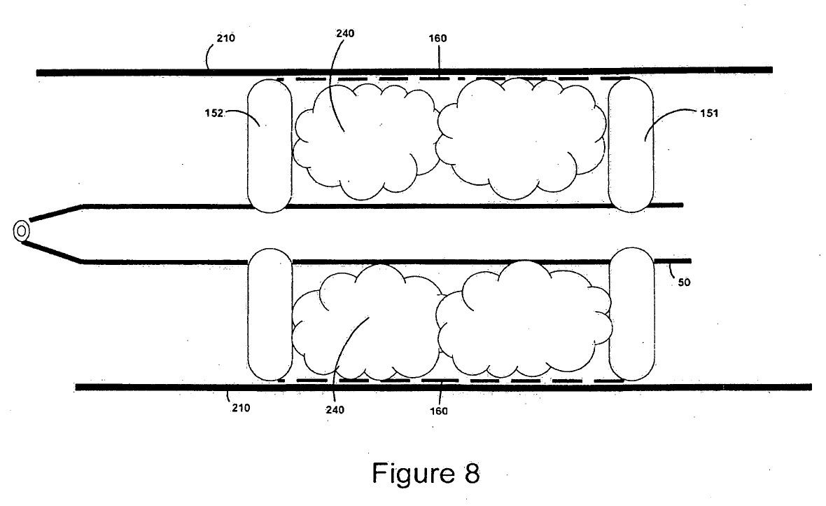

FIG. 8 illustrates a cross sectional view of the fully deployed net 160 extending to the wall 210 of the artery. The balloons 151,152 are fully deployed. The device is dimensioned to meet the interior diameter of the vessel wall 210. The fenestrations 165 of the net are also illustrated. The embolus is shown trapped between the net and the catheter 50.

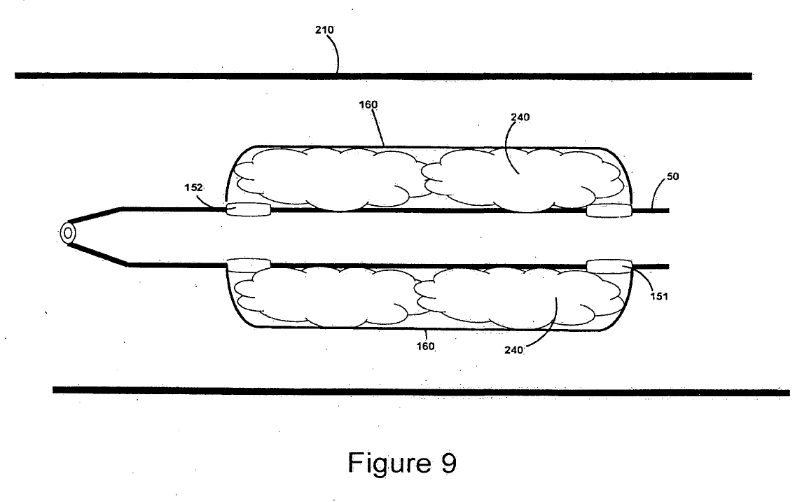

FIG. 9 is a cross sectional view of the start of the retraction process with the balloons 151,152 fully deflated. The diameter of the net 160 is smaller and has retreated from the artery wall 210. Carried within the net is the entrapped embolus 240. Retraction of the catheter 50 withdraws the device from the artery.

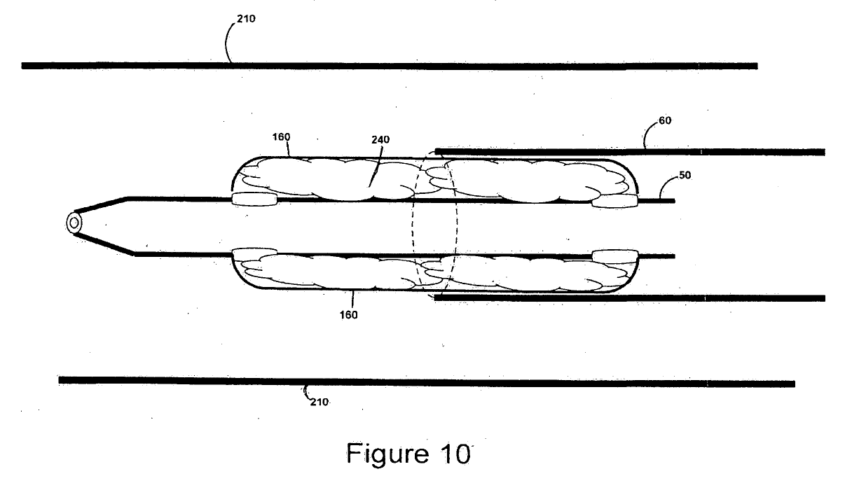

FIG. 10 illustrates the device (described in FIG. 9) entering the guiding catheter 60. At least a portion of an embolus may be removed by deploying the first catheter, with its segments contracted and the net folded up on the shaft of the catheter and the contracted segments, along a guide wire into the embolus. The proximal inflatable segment (the balloon) is positioned just proximal to the clot using the radio opaque marker on the balloon catheter. Once positioned in the embolus, the balloon may be expanded, thereby expanding and unfolding the cone ended tubular shaped net or expanding, stretching and unfolding the body of the net. The net may be expanded to unfold and deploy the net through and around the clot, toward the walls of the artery. The clot will be squeezed between the walls of the artery and the net forcing the clot through the fenestrations and into the net, then deflate the balloons either partially or completely to collapse the net on the clot, capturing a substantial amount of the embolus between the balloon segments and the net. The entrapment of the clot material within the net may be enhanced by use of the suction ports within the first catheter, creating a negative pressure within the area within the porous net. The first catheter is then withdrawn from the occluded branch and removing the contracted balloon, net and the entrapped embolus between the catheter and net into the guiding catheter.

In an additional embodiment, the device may comprise a first catheter containing a net at the distal end. A separate second catheter fitted with fluid, inflating balloons and dimensioned to fit within the first catheter is used to inflate the net. This second catheter may contain one or more suction ports used to draw embolic material through the net. These ports are connected to tubing in communication with a device generating negative pressure.

Thus, embodiments described herein comprise a combination of features and advantages intended to address various shortcomings associated with certain prior devices. The various characteristics described above, as well as other features, will be readily apparent to those skilled in the art upon reading the following detailed description of the preferred embodiments, and by referring to the accompanying drawings. It should be appreciated by those skilled in the art that the conception and the specific embodiments disclosed may be readily utilized as a basis for modifying or designing other structures for carrying out the same purposes of the embodiments described herein. It should also be realized by those skilled in the art that such equivalent constructions do not depart from the spirit and scope of the invention.

What I claim is:

1. A net expanded by two or more inflatable balloons affixed to a first catheter that can be guided through the tortuous and stenotic cerebral circulatory system, thereby ensnaring an embolus material with the net containing fenestrations and deflating the balloons to compress the volume of the net and retracting the catheter with the collapsed net and deflated balloons.

2. The device of claim 1 further comprising a guiding catheter into which the first catheter balloon and net may be retracted.

3. The device of claim 1 further comprising a tubular shaped net containing fenestrations and sized to fit within a cerebral vessel.

4. The device of claim 3 further comprising fenestrations distributed around the circumference of the tubular shaped net.

5. The device of claim 3 further comprising fenestrations in linear shape, oval shape, rectangular shape, serpentine shape, parallelogram shape, or diamond shape.

6. The device of claim 3 further comprising a fenestration that has an aperture for passage of an embolic material when the balloons are inflated and circumference of the net is expanded and when the balloon is deflated, the circumference of the net decreases and the aperture is essentially closed.

7. A method for removing at least a portion of an embolus comprising:
a) placing a guide wire proximate to the site of the embolus within a cerebral vessel;
b) maneuvering a first catheter along the guide wire through the tortuous and stenotic cerebral circulatory system and through the embolus;
c) transporting two or more inflatable balloons and a net by the first catheter;
d) inflating the balloons with fluid through the first catheter and controlling the radial expansion of the net;
e) using the radial, expansion of the net to entrap the embolus within the net through net fenestrations;
f) deflating the balloons thereby entrapping embolic material in the net; and
g) withdrawing the first catheter including balloons and net.

8. A method of removing an embolus comprising
a) maneuvering a catheter carrying at least two balloons and a net through the tortuous and stenotic cerebral blood circulatory system;
b) positioning the catheter at the embolism;
c) inflating the balloons causing deployment of the net with fenestrations;
d) continuing inflation of the balloon so that the net is proximate to the vessel wall;
e) entrapping the embolus within the net:
f) deflating the balloon and collapsing the net now containing entrapped embolus material; and
g) withdrawing the catheter.

9. The method of claim 8 further comprising withdrawing the catheter into a second guiding catheter.

10. An embolus removing net material comprising:
a) woven or non woven flexible and biocompatible small diameter micro threads; and
b) wherein the small diameter micro threads are non-elastic or non-compliant.

11. The material of claim 10 further comprising small diameter micro threads that are semi-compliant within a middle section between inflatable balloons.

12. The material of claim 10 further comprising the small diameter micro-threads arranged in a helically wound pattern.

13. The material of claim 10 further comprising the small diameter micro-threads arranged in a braided pattern.

14. The material of claim 10 further comprising the small diameter micro-threads arranged parallel to a longitudinal axis of the catheter.

15. An embolus removing net material comprising a tubular shape with cone shaped ends wherein the outer diameter of the tubular shaped net material is approximately equal to the interior diameter of a vessel containing an embolus.

16. The net material of claim 15 further comprising the cone shaped ends fused to the catheter.

17. The net material of claim 15 further comprising each cone shaped end fused to a balloon.

18. The net material of claim 15 further comprising small diameter micro threads further comprising at least one extended thread that when pulled reduces the diameter of the tubular portion of the net material.

19. The net material of claim 15 further comprising attaching each net material end separately to the catheter.

20. The net material of claim 15 further comprising fenestrations within the material between the two ends that are round shaped or diamond shaped.

21. The net material of claim 15 further comprising fenestrations within the material between the two ends that are oval or rectangular shaped.

22. The net material of claim 15 further comprising fenestrations that are opened by pushing on a mechanism attached to a first catheter and closed by pulling upon the mechanism attached to the first catheter.

23. The net material of claim 15 further comprising fenestrations that are opened by stretching the said material with at least one inflatable balloon and closed with deflating the balloon.

24. An embolus removing system comprising:
a) at least two compliant balloons attachable to a catheter; and
b) a substantially non compliant net component further comprising fenestrations that can be controllably opened and closed.

25. An embolus removing system comprising:
a) attaching a substantially non compliant net material to a catheter and further comprising fenestrations in the net material that can be controllably opened or closed;
b) inserting the net and catheter within the embolus;
c) inserting inflatable balloon components through the catheter proximate to the net component; and
d) inflating the balloons to open the fenestrations of the net material.
